(19) United States
(12) Patent Application Publication
(10) Pub. No.: US 2025/0134415 A1
(43) Pub. Date: May 1, 2025

(54) AMBIENT ABILITY ASSESSMENT AND CARE MANAGEMENT

(71) Applicant: Welch Allyn, Inc., Skaneateles, NY (US)

(72) Inventors: WonKyung MCSWEENEY, Manlius, NY (US); David E. QUINN, Auburn, NY (US); Gene J. WOLFE, Pittsford, NY (US); John A. LANE, Venice, FL (US); Tyson B. WHITAKER, Arden, NC (US)

(21) Appl. No.: 18/930,109

(22) Filed: Oct. 29, 2024

Related U.S. Application Data
(60) Provisional application No. 63/594,998, filed on Nov. 1, 2023.

(51) Int. Cl. A61B 5/11; A61B 5/00; G16H 40/67

(52) U.S. Cl. CPC A61B 5/1124; A61B 5/0004; A61B 5/447; A61B 5/7275; G16H 40/67

(57) ABSTRACT

A method for monitoring subject ability comprises using a transmitting device, to transmit a signal from a subject performing a task, using a receiving device to acquire data transmitted from the signal, processing the data by an ability analysis device. The ability analysis device is configured to calculate a subject ability score. A treatment protocol is identified based on the subject ability score, and the subject ability score and the treatment protocol is communicated to a user device. The subject is treated with the identified treatment protocol.

### CROSS-REFERENCE TO RELATED APPLICATIONS

This application claims priority under 35 U.S.C. § 119(e) to U.S. Provisional Application No. 63/594,998, filed Nov. 1, 2023, which is expressly incorporated by reference herein.

### BACKGROUND

The present disclosure relates to the use of tools and techniques for continuously monitoring a subject to identify and determine a level of subject ability to be independent, changes in subject ability over time, and appropriate treatments based on the determined level of subject ability.

It is important to determine subject's ability to move and independently perform tasks in the subject room. Movement in this context means a change in position of the subject on the subject support apparatus, relative to other components in the subject room, and as relevant to subject ability to perform tasks. A subject's ability to be independent may be indicative of a subject's health and/or medical needs. Additionally, assessing the independence level of subjects is a critical to determine their ability to perform daily activities and move around. Accurate assessment of this ability level helps healthcare providers develop a care plan that can caters to the subject's needs.

Further, an assessment of subject ability to move can be instrumental in preventing subject falls, pressure ulcers, and other complications associated with immobility. Subject ability information can provide valuable information for discharge, planning, and subject rehabilitation. Subject falls, pressure ulcers, and inaccurate discharge planning can lead to significant issues that increase healthcare costs. According to a study by the Agency for Healthcare Research and Quality (AHRQ), subject falls alone cost hospitals an estimated $50 billion annually in the United States. Additionally, pressure ulcers can result in longer hospital stays and increased treatment costs, ranging from $9.1 billion to $11.6 billion per year. Inaccurate discharge planning can also lead to readmissions, which can be expensive. A study published in the Journal of Hospital Medicine found that unplanned readmissions within 30 days of discharge cost hospitals an average of $14,400 per subject.

Performing and monitoring the ability of subjects to be independent and/or self-sufficient can be challenging due to various factors such as subject cooperation, staffing shortages, and limited resources. Subjects may be uncooperative or unable to follow instructions, making it difficult to assess their ability level accurately. Staffing shortages and limited resources can also make it challenging to monitor subjects' ability level consistently.

Thus, there is a need to improve the systems and methods used for monitoring and assessing subject ability in healthcare systems. Characterizing a subject's ability and calculating a subject's ability score can improve subject care and the determination of future treatment protocols or procedures.

### SUMMARY

The present disclosure includes one or more of the features recited in the appended claims and/or the following features which, alone or in any combination, may comprise patentable subject matter.

According to a first aspect of the present disclosure, a method for monitoring subject ability comprises the steps of transmitting, using a transmitting device, a signal from a subject performing a task, the signal indicating subject ability to be independent, acquiring, using a receiving device, data transmitted from the signal, processing the data by an ability analysis device, the ability analysis device configured to calculate a subject ability score, identifying a treatment protocol based on the subject ability score, and outputting the subject ability score and the treatment protocol to a user device.

In some embodiments, the method further comprises identifying an assistive device in a vicinity of the subject, and detecting use of the assistive device by the subject when the subject is performing the task. In some embodiments, the method further comprises identifying a helper in a vicinity of the subject, and detecting assistance provided by the helper when the subject is performing the task.

In some embodiments, the method further comprises determining if the helper is a caregiver or a member of the subject family. In some embodiments, the method further comprises using the assistive device to track contact points to predict pressure ulcers.

In some embodiments, if the subject does not use the assistive device and does not use the assistance provided by the helper to perform the task, the subject is assigned a first ability score. In some embodiments, if the subject uses the assistive device and does not use the assistance provided by the helper to perform the task, the subject is assigned a second ability score lower than the first ability score. In some embodiments, if the assistance provided by the helper is for safety purposes and the subject independently performs about 100% of the task, the subject is assigned a third ability score lower than the second ability score.

In some embodiments, if the assistance provided by the helper is for light touching and the subject independently performs about 100% of the task, the subject is assigned a fourth ability score lower than the third ability score. In some embodiments, if the subject independently performs more than a first threshold percentage of the task and less than about 100% of the task, the subject is assigned the fourth ability score lower than the third ability score.

In some embodiments, if the subject independently performs more than a second threshold percentage of the task and less than the first threshold percentage of the task, the subject is assigned a fifth ability score lower than the fourth ability score. In some embodiments, if the subject independently performs more than a third threshold percentage of the task and less than the second threshold percentage of the task, the subject is assigned a sixth ability score lower than the fifth ability score. In some embodiments, if the subject independently performs less than third threshold percentage of the task, the subject is assigned a seventh ability score lower than the sixth ability score.

In some embodiments, the transmitting device comprises a wrist band. In some embodiments, the transmitting device is comprised in a subject gown. In some embodiments, the transmitting device is a reflector located on a subject apparatus. In some embodiments, the receiving device is a video camera.

In some embodiments, the ability analysis device is configured to determine a motion of the subject by detecting a first set of body points at a first time and a second set of body points at a second time, and tracking changes between the first set of body points and the second set of body points to detect subject ability. In some embodiments, the ability analysis device is configured to determine a range of motion of the subject before calculating the subject ability score. In some embodiments, the ability analysis device is configured to determine a speed of motion of the subject before calculating the subject ability score. In some embodiments, the method further comprises performing the treatment protocol.

According to a second aspect of the present disclosure, a system for monitoring subject ability comprises a transmitting device for transmitting a signal from a subject performing a task, the signal indicating subject ability to be independent, a receiving device for acquiring data from the signal, an ability analysis device for processing the data, the ability analysis device configured to calculate a subject ability score and to identify a treatment protocol based on the subject ability score, and a user device for outputting the subject ability score and the treatment protocol to a user.

In some embodiments, the system further comprises an assistive device in a vicinity of the subject, the assistive device configured to transmit information to the ability analysis device. In some embodiments, the ability analysis device is configured to identify a helper in a vicinity of the subject, and to detect assistance provided by the helper when the subject is performing the task. In some embodiments, the ability analysis device is configured to determine if the helper is a caregiver or a member of the subject family. In some embodiments, the assistive device is configured to track contact points to predict pressure ulcers on the subject.

In some embodiments, the ability analysis device is configured to assign a first ability score to the subject if the subject does not use the assistive device and does not use the assistance provided by the helper to perform the task. In some embodiments, the ability analysis device is configured to assign a second ability score less than the first ability score to the subject if the subject uses the assistive device and does not use the assistance provided by the helper to perform the task. In some embodiments, the ability analysis device is configured to assign a third ability score less than the second ability score to the subject if the assistance provided by the helper is for safety purposes and the subject independently performs about 100% of the task.

In some embodiments, the ability analysis device is configured to assign a fourth ability score less than the third ability score to the subject if the assistance provided by the helper is for light touching and the subject independently performs about 100% of the task. In some embodiments, the ability analysis device is configured to assign the fourth ability score less than the third ability score the subject independently performs more than a first threshold percentage of the task and less than about 100% of the task.

In some embodiments, the ability analysis device is configured to assign a fifth ability score less than the fourth ability score to the subject if the subject independently performs more than a second threshold percentage of the task and less than the first threshold percentage of the task. In some embodiments, the ability analysis device is configured to assign a sixth ability score less than the fifth ability score to the subject if the subject independently performs more than a third threshold percentage of the task and less than the second threshold percentage of the task. In some embodiments, the ability analysis device is configured to assign a seventh ability score less than the sixth ability score to the subject if the subject independently performs less than the third threshold percentage of the task.

In some embodiments, the transmitting device comprises a wrist band. In some embodiments, the transmitting device is comprised in a subject gown. In some embodiments, the transmitting device is a reflector located on a subject apparatus. In some embodiments, the receiving device is a video camera.

In some embodiments, the ability analysis device is configured to determine a motion of the subject by detecting a first set of body points at a first time and a second set of body points at a second time, and tracking changes between the first set of body points and the second set of body points to detect subject ability to be independent. In some embodiments, the ability analysis device is configured to determine a range of motion of the subject before calculating the subject ability score. In some embodiments, the ability analysis device is configured to determine a speed of motion of the subject before calculating the subject ability score.

According to a third aspect of the present disclosure, an ability analysis device is configured to process ability data of a subject obtained from a receiving device, calculate a subject ability score of the subject based on the processed data, and output the subject ability score to a user device.

In some embodiments, the receiving device is configured to receive the data from a transmitting device. In some embodiments, the transmitting device is configured to identify a subject performing a task. In some embodiments, the device is further configured to communicate with an assistive device in a vicinity of the subject, the assistive device configured to transmit information to the ability analysis device. In some embodiments, the ability analysis device is further configured to identify a helper in a vicinity of the subject, and to detect assistance provided by the helper when the subject is performing the task.

In some embodiments, the ability analysis device is configured to determine if the helper is a caregiver or a member of the subject family. In some embodiments, the assistive device is configured to track contact points to predict pressure ulcers on the subject.

In some embodiments, the ability analysis device is configured to assign a first ability score to the subject if the subject does not use the assistive device and does not use the assistance provided by the helper to perform the task. In some embodiments, the ability analysis device is configured to assign a second ability score less than the first ability score to the subject if the subject uses the assistive device and does not use the assistance provided by the helper to perform the task. In some embodiments, the ability analysis device is configured to assign a third ability score less than the second ability score to the subject if the assistance provided by the helper is for safety purposes and the subject independently performs about 100% of the task.

In some embodiments, the ability analysis device is configured to assign a fourth ability score less than the third ability score to the subject if the assistance provided by the helper is for light touching and the subject independently performs about 100% of the task. In some embodiments, the ability analysis device is configured to assign the fourth ability score less than the third ability score to the subject if the subject independently performs more than a first threshold percentage of the task and less than about 100% of the task. In some embodiments, the ability analysis device is configured to assign a fifth ability score less than the fourth ability score to the subject if the subject independently performs more than a second threshold percentage of the task and less than the first threshold percentage of the task.

In some embodiments, the ability analysis device is configured to assign a sixth ability score less than the fifth ability score to the subject if the subject independently performs more than a third threshold percentage of the task and less than the second threshold percentage of the task. In some embodiments, the ability analysis device is configured to assign a seventh ability score less than the sixth ability score to the subject if the subject independently performs less than the third threshold percentage of the task.

In some embodiments, the transmitting device comprises a wrist band. In some embodiments, the transmitting device is comprised in a subject gown. In some embodiments, the transmitting device is a reflector located on a subject apparatus. In some embodiments, the receiving device is a video camera.

In some embodiments, the device is configured to determine a motion of the subject by detecting a first set of body points at a first time and a second set of body points at a second time, and tracking changes between the first set of body points and the second set of body points to detect subject ability. In some embodiments, the ability analysis device is configured to determine a range of motion of the subject before calculating the subject ability score. In some embodiments, the ability analysis device is configured to determine a speed of motion of the subject before calculating the subject ability score.

According to a fourth aspect of the present disclosure, an method for monitoring subject ability comprising the steps of transmitting, a indicating subject ability to be independent, acquiring data transmitted from the signal, processing the data to calculate a subject ability score, identifying a treatment protocol based on the subject ability score, and outputting the subject ability score and the treatment protocol inform a subject treatment.

In some embodiments, the method further comprises identifying an assistive device in a vicinity of the subject, and detecting use of the assistive device by the subject when the subject is performing the task.

In some embodiments, the method further comprises identifying a helper in a vicinity of the subject, and detecting assistance provided by the helper when the subject is performing the task.

In some embodiments, the method further comprises determining if the helper is a caregiver or a member of the subject family. In some embodiments, the method further comprises using the assistive device to track contact points to predict pressure ulcers.

In some embodiments, if the subject does not use the assistive device and does not use the assistance provided by the helper to perform the task, the subject is assigned a first ability score. In some embodiments, if the subject uses the assistive device and does not use the assistance provided by the helper to perform the task, the subject is assigned a second ability score lower than the first ability score. In some embodiments, if the assistance provided by the helper is for safety purposes and the subject independently performs about 100% of the task, the subject is assigned a third ability score lower than the second ability score. In some embodiments, if the assistance provided by the helper is for light touching and the subject independently performs about 100% of the task, the subject is assigned a fourth ability score lower than the third ability score.

In some embodiments, if the subject independently performs more than a first threshold percentage of the task and less than about 100% of the task, the subject is assigned the fourth ability score lower than the third ability score. In some embodiments, if the subject independently performs more than a second threshold percentage of the task and less than the first threshold percentage of the task, the subject is assigned a fifth ability score lower than the fourth ability score. In some embodiments, if the subject independently performs more than a third threshold percentage of the task and less than the second threshold percentage of the task, the subject is assigned a sixth ability score lower than the fifth ability score. In some embodiments, if the subject independently performs less than third threshold percentage of the task, the subject is assigned a seventh ability score lower than the sixth ability score.

In some embodiments, the method further comprises performing the treatment protocol.

### BRIEF DESCRIPTION OF THE DRAWINGS

The detailed description particularly refers to the accompanying figures in which:

FIG. 1 is an illustration of an ability monitoring system comprising different components.

FIG. 2 is an illustration of one embodiment of an ability analysis device communicating with other components of the ability monitoring system.

FIG. 3 is a flowchart illustrating a process implemented by the ability monitoring system to identify and/or detect subject motion.

FIG. 4 is a flowchart illustrating the classification of subject ability and the assignment of an ability score by the ability analysis device.

### DETAILED DESCRIPTION

To solve the challenges in assessing a healthcare subject's ability level, the invention implements an ambient ability monitoring system. The healthcare subject may be a patient, a person in an assisted living facility, an athlete, or any person needing the use of a healthcare facility. Patient and subject are used interchangeably herein to identify a user of an ability monitoring system.

At the subject's admission, or at any other triggered time point, the ability monitoring system can start observing the subject's movement when the subject is performing a task. The ability monitoring system can also monitor the presence and use of helpers and assistive devices in the subject vicinity to calculate an ability score. The ability monitoring system is operated in the background without prompting the subject, and continues to monitor the subject even when a caregiver is not present. The ability monitoring system is configured to monitor when the subject needs to move, proving a healthcare system with realistic assessment of subject ability level while also freeing the caregiver resources typically used for such assessment.

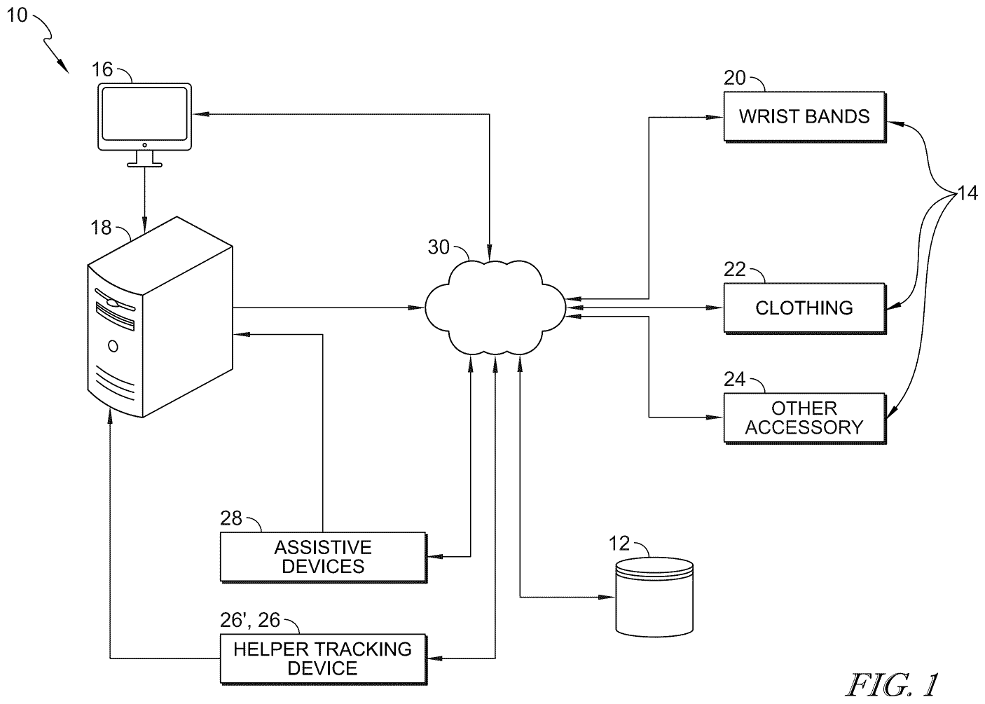

According to the present disclosure, an ability monitoring system 10 can implemented to provide continuous and real-time analysis of subject ability. The ability monitoring system 10 can be implemented for detecting, reporting, analyzing, and/or tracking subject motion in a healthcare system. An illustrative embodiment of the ability monitoring system 10 is shown in FIG. 1. The ability monitoring system 10 utilizes data 12 captured by one or more transmitting devices 14 to inform subject movement. The data 12 from the transmitting devices 14 is acquired by a receiving device 16 and eventually processed by an ability analysis device 18 or an ability analyzer 18. The ability analysis device 18 can be a control system 18, a special purpose device 18, or a controller 18 associated with another component of the ability monitoring system 10. The ability analysis device 18 can be a discrete device, or a more general-purpose computing system, a smart device, a personal device, or similar, embodying the disclosed and claimed features and functions of the device as a part thereof. For example, the ability analysis device 18 can be an application (“app”) in a cell phone or in a computing device.

The ability analysis device 18 can implement machine learning and/or artificial intelligence be to determine a subject ability score based in part on the assessed subject motion. In some embodiments, the ability analysis device 18 may be configured to determine a range of motion of the subject. In some embodiments, the ability analysis device 18 may be configured to determine a speed of motion of the subject. In some embodiments, the ability analysis device 18 may be configured to determine subject independence or subject self-sufficiency when the subject is engaging in performing tasks. The phrase “to be independent” and more generally subject independence are used broadly herein to connote a discrete, stepwise, and/or continuous indicator of a subject's ability to perform e.g., a task, physical activity, movement, and the like.

Smart technologies can be implemented to enable continuous monitoring of subject movement, assess changes in subject movement, and determine treatment protocols or procedures based on the assessed subject movement. This real-time information can be imported into a subject medical record by the ability monitoring system 10. The ability monitoring system 10 can be utilized before performing a subject treatment. The ability monitoring system 10 can be utilized for short-term post-event analysis and for long-term post-event analysis.

The ability monitoring system 10 uses information received from the various transmitting devices 14 including but not limited to a wrist band 20, a clothing accessory 22, or any other identifying accessory 24 over a network 30. In some embodiments, the transmitting device 14 may be a RFID tag 24 that could be identified by an RFID reader connected to the receiving device 16.

In one embodiment, the receiving device 16 can be a video monitoring system 16 such as a video camera 16. The receiving device 16 can identify the subject based on the signal emitted from the transmitting device 14. In some embodiments, the receiving device 16 can identify the subject based on visual identification of the transmitting device 14. In some embodiments, the ability monitoring system 10 can comprise more than one receiving device 16. Additionally, the ability analysis device 18 can receive information from a helper tracking device 26 and/or one or more assistive devices 28. In some embodiments, additional user input may be provided to the ability analysis device 18 to determine the ability score.

The assistive devices 28 can include a subject support device 28, such as a bed, on which the subject can lie, rest, or sleep. Other examples of the assistive devices 28 include lifts, chairs, stretchers, and surgical tables. In this document, the subject support device 28 is also referred to as the bed 28. The ability analysis device 18 can receive information from one or more assistive devices 28 in the vicinity of the subject. In some embodiments, the vicinity of the subject can be defined by the caregiver. In other embodiments, the vicinity of the subject can be the entire subject room.

The helper tracking devices 26 can be provided to a helper 26′ who is available to help the subject perform his tasks. The helper tracking devices 26 can be used to distinguish the type of helper 26′ and identify if the helper 26′ is a caregiver or a member of the subject family. The helper tracking device 26 can be a wrist band, a clothing accessory, or a badge.

Examples of the helper 26′ include a parent of the subject, a family member of the subject, a caregiver of the subject, a primary physician of the subject, and any other interested parties. The caregiver is a person who provides healthcare service to the subject. Examples of caregiver include primary care providers (e.g., doctors, nurse practitioners, and physician assistants), nursing care providers (e.g., nurses), specialty care providers (e.g., professionals in various specialties), and health professionals that provide preventive, curative, promotional and rehabilitative health care services. Although the system 10 is primarily described with respect to a single subject, it is understood that a plurality of subjects can be monitored with the system of the present disclosure, either individually or in group.

The ability monitoring system 10 of the present disclosure operates to detect a current status of a subject in a subject arrangement area, such as in a room, and determine the status of subject ability. The transmitting devices 14 may use a signal to transmit data 12. In certain examples, the signal is transmitted toward the receiving device 16 and is used to detect a subject status, such as a subject position, a subject contact condition, a subject ambulation, and/or a physiological condition of the subject. One embodiment of the signal includes a millimeter wave, such as a signal having a band of spectrum between abound 30 GHz and about 300 GHz. Other embodiments of the signal include microwaves and ultrawideband (UWB) or optical waves. The ability analysis device 18 can acquire the data 12 from the receiving device 16 and process the data 12 to determine the subject ability score.

In certain embodiments, the ability analysis device 18 can detect one or more body portions of the subject by analyzing the information acquired from the receiving device 16 and predicting an upcoming position of the subject based on the analysis. For example, the ability analysis device 18 can determine if the subject achieved his task independently or used the assistance of a helper 26′ and/or an assistive device 28. In another embodiment, the ability analysis device 18 can operate to predict a likelihood that a subject falls from a subject bed based on the arrangement of detected body positions and previously calculated subject ability score. The ability monitoring system 10 can generate a notification to a caregiver so that the caregiver can assist the subject or prevent the subject from falling of the bed.

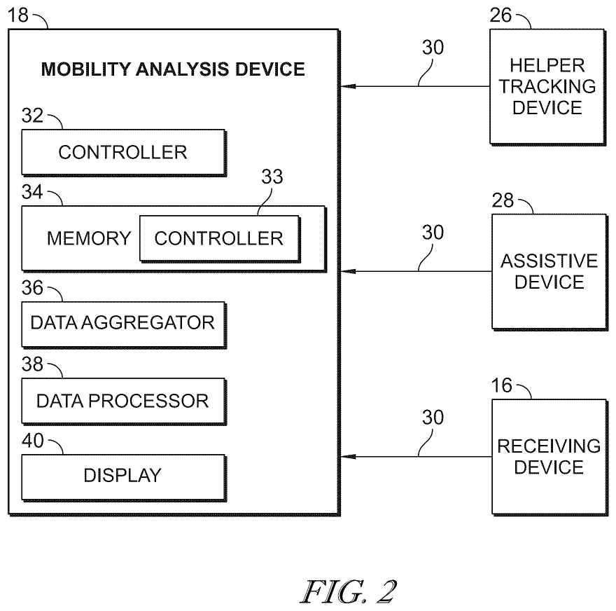

FIG. 2 illustrates one embodiment of the ability analysis device 18. The ability analysis device 18 can receive information from the receiving devices 14, the assistive devices 28, and the helper tracking devices 26 to determine the subject ability score. A processor 38 and a data aggregator 36 can process and analyze the information received from the receiving devices 14. The processor 38 and memory device 34 are shown only with respect to the ability analysis device 18, but similar structures are used in the tracking devices 26, the assistive devices 28, the receiving devices 14, and the transmitting devices 14.

The memory device 34 includes instructions 33 that, when executed by the processor 38, cause the processor 38 and controller 32 to perform functions as associated with the ability analysis device 18. It should be understood that the term processor here includes any microprocessor, microcontroller, processor circuitry, control circuitry, preprogrammed device, or any structure capable of accessing the memory device and executing non-transient instructions to perform the tasks, algorithm, and process disclosed herein. In the illustrative embodiment, the ability analysis device 18 employs a conventional controller area network (CAN) for communications between subsystems, but it should be understood that any of a number of networking and communications solutions could be employed in the ability analysis device 18.

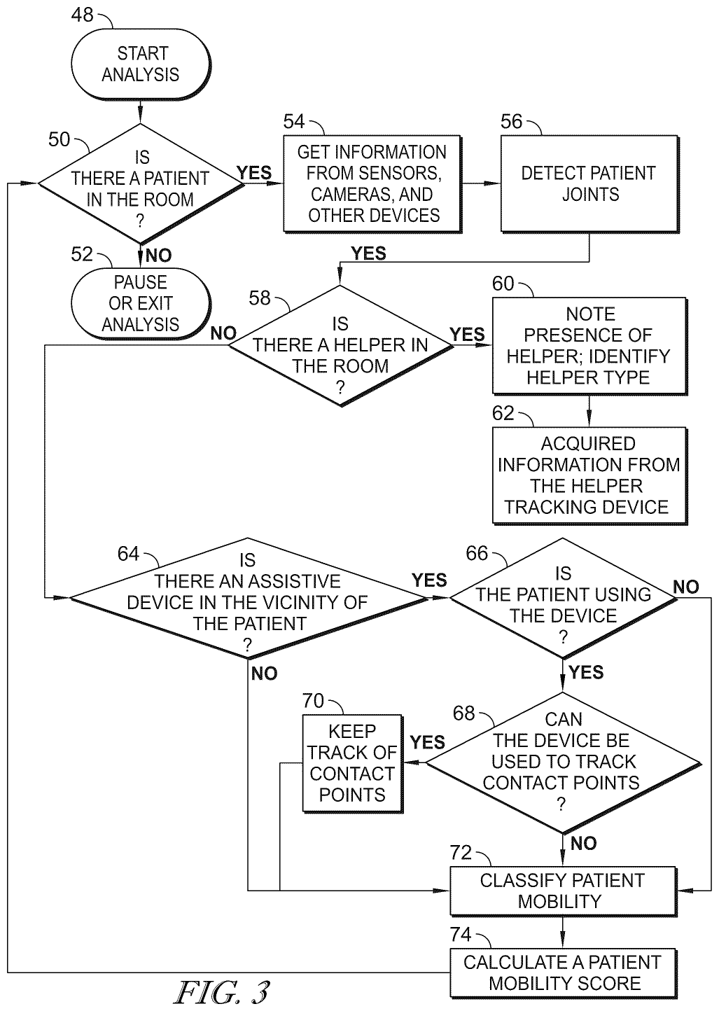

FIG. 3 illustrates a decision-making flowchart implemented by the ability monitoring system 10 to identify and/or detect subject motion. The ability monitoring system 10 can begin the subject ability analysis when the subject enters a certain room, and/or a certain region of the healthcare system. Since the ability monitoring system 10 provides continuous monitoring of subject ability, the ability analysis device 18 may be continuously monitoring for the presence of the subject in step 48. If the receiving device 16 does not detect a subject in step 50, the subject ability analysis is paused or stopped. If the receiving device 16 detects a subject in step 50, the receiving device 16 acquires signals from one or more transmitting devices 14 and the ability analysis device 18 acquires and process the data from the receiving device in step 54. Further, the ability analysis device 18 may detect location of subject joints in step 54 based on the information acquired from the receiving device.

The ability analysis device 18 can be configured to operate intermittently and evaluate the subject vicinity in fixed time intervals. Alternatively, or additionally, the ability analysis device 18 can be configured to be triggered by specific triggering events. The triggering events can include any subject movement, or pre-defined events including but not limited to eating, bathing, and turning on subject apparatus. Once triggered, the ability analysis device 18 can be configured to monitor the subject for a fixed interval of time or until the ability analysis device 18 determines that the subject has stopped performing the triggering task.

The ability analysis device 18 determines the presence of any helper 26′ in step 58. If a helper 26′ is not detected, the ability analysis device 18 determines the presence of any assistive device 28 in step 64. If a helper 26′ is detected in step 58, the ability analysis device 18 notes the presence of the helper 26′ and the helper type (subject family or caregiver) in step 60. Further, the ability analysis device 18 acquires information form the helper tracking device 26 in step 62. Information can include information about proximity of the helper 26′ to the subject, information about time spent by the helper 26′ in the vicinity of the subject, etc. Once the information is obtained from the helper tracking device 26, the ability analysis device 18 determines the presence of any assistive device 28 in step 64.

If the assistive device 28 is not detected, the ability analysis device 18 classifies subject activity in step 72. If the assistive device 28 is detected is step 64, the ability analysis device 18 determines if the subject is using the assistive device 28 in step 66. If the assistive device 28 is not using the assistive device 28, the ability analysis device 18 classifies subject activity in step 72. If the subject is using the assistive device 28, the ability analysis device 18 determines if the assistive device 28 can be used to track contact points on the subject in step 68. If the assistive device 28 cannot be used to track contact points on the subject, the ability analysis device 18 classifies subject activity in step 72. If the assistive device 28 can be used to track contact points on the subject, the ability analysis device 18 keeps track of the contact points for a given time period to prevent formation of pressure ulcers and then classifies subject activity in step 72.

The ability analysis device 18 classifies subject ability in step 72 based on the amount of assistance the subject uses to perform a given task. The assistance may be from a helper tracking device 26 or the assistive device 28. Subject is then assigned an ability score based on the classification in step 74.

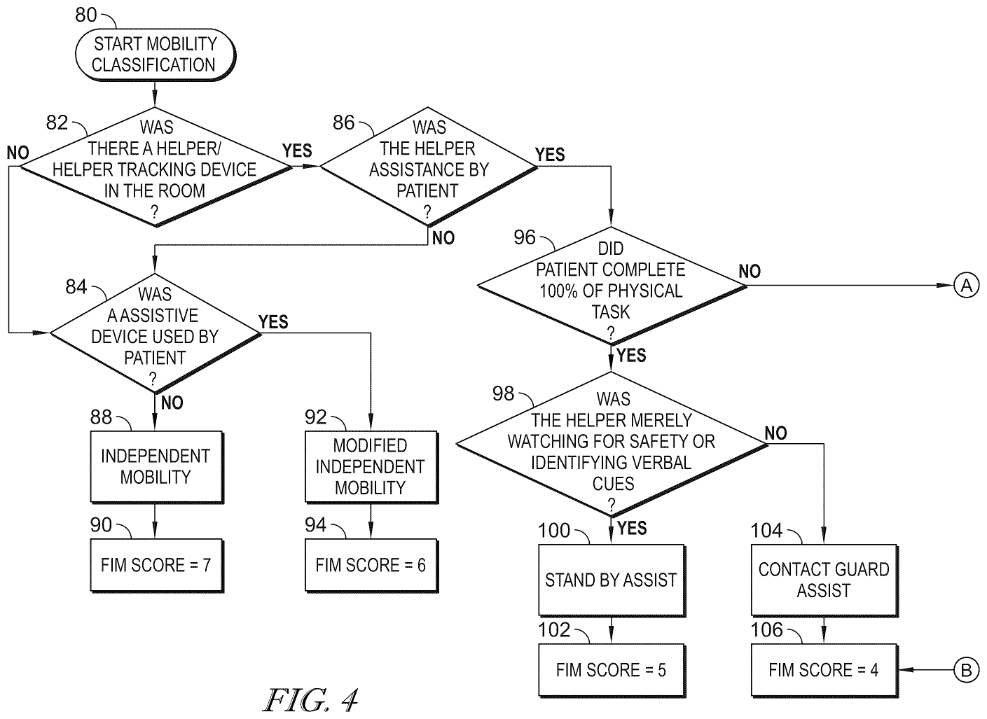

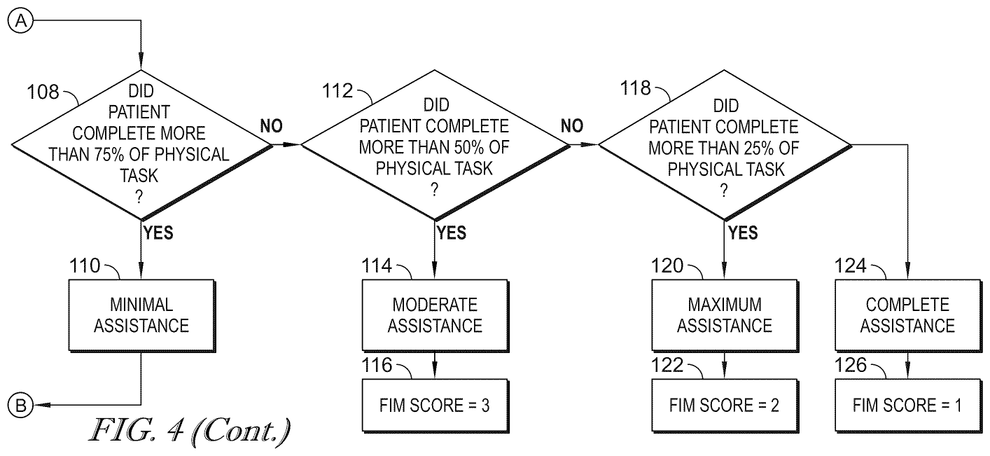

FIG. 4 illustrates the classification of subject ability and the assignment of the ability score by the ability analysis device 18. When ability classification is initiated in step 80, the ability analysis device 18 is configured to determine if the helper tracking device 26 was detected in the room in step 82. If no helper tracking device 26 was detected, the ability analysis device 18 is configured to determine if an assistive device 28 was used by the subject in step 84. If the helper tracking device 26 was detected in step 82, the ability analysis device 18 is configured to determine if the helper 26′ assisted the subject in performing his task in step 86. If the helper 26′ did not assist the subject in performing his task, the ability analysis device 18 is configured to determine if an assistive device 28 was used by the subject in step 84. The ability analysis device 18 can also determine if the helper 26′ is a caregiver or a member of the subject's family.

If no assistive device 28 was used by the subject, as determined in step 84, the ability analysis device 18 is configured to classify the subject ability as ‘independent ability’ in step 88, and the subject is assigned a first ability score, which is indicative of least independence in step 90. In some embodiments, the first ability score is 7. If an assistive device 28 was used by the subject, as determined in step 84, the ability analysis device 18 is configured to classify the subject ability as ‘modified independent ability’ in step 92, and the subject is assigned a second ability score, which is lower than the first ability score in step 94. In some embodiments, the second ability score is 6.

If the helper 26′ is determined to help the subject perform his task in step 86, the ability analysis device 18 is further is configured to determine if the subject completed about 100% of the physical task in step 96 and if the helper 26′ is merely watching for safety or providing verbal cues in step 98. If so, the ability analysis device 18 is configured to classify the subject ability as ‘standby assist’ in step 100, and the subject is assigned a third ability score, which is lower than the second ability score in step 102. In some embodiments, the third ability score is 5.

If the ability analysis device 18 determines that the subject did complete about 100% of the physical task in step 96, but the helper 26′ is not merely watching for safety or providing verbal cues in step 98 the ability analysis device 18 is configured to classify the subject ability as ‘contact guard assist’ in step 104, and the subject is assigned a fourth ability score, which is lower than the third ability score in step 106. In some embodiments, the fourth ability score is 4.

If the ability analysis device 18 determines that the subject did not complete 100% of the physical task in step 96, the ability analysis device 18 is further configured to determine if the subject completed more than a first threshold percentage of the physical task in step 108. If the ability analysis device 18 determines that the subject did complete more than about the first threshold percentage of the physical task in step 108, the ability analysis device 18 is configured to classify the subject ability as ‘minimal assistance’ in step 110, and the subject is assigned the fourth ability score, which is lower than the third ability score in step 106. In some embodiments, the first threshold percentage is about 75%. In some embodiments, the first threshold percentage is in the range of about 60% to about 80%, including any percentage comprised therein.

If the ability analysis device 18 determines that the subject did not complete more than the first threshold percentage of the physical task in step 108, the ability analysis device 18 is further configured to determine if the subject completed more than a second threshold percentage of the physical task in step 112. If the ability analysis device 18 determines that the subject did complete more than the second threshold percentage of the physical task in step 112, the ability analysis device 18 is configured to classify the subject ability as ‘moderate assistance’ in step 114, and the subject is assigned a fifth ability score, which is lower than the fourth ability score in step 116. In some embodiments, the fifth ability score is 3. In some embodiments, the second threshold percentage is about 50%. In some embodiments, the second threshold percentage is in the range of about 30% to about 60%, including any percentage comprised therein.

If the ability analysis device 18 determines that the subject did not complete more than the second threshold percentage of the physical task in step 112, the ability analysis device 18 is further configured to determine if the subject completed more than a third threshold percentage of the physical task in step 118. If the ability analysis device 18 determines that the subject did complete more than the third threshold percentage of the physical task in step 118, the ability analysis device 18 is configured to classify the subject ability as ‘maximum assistance’ in step 120, and the subject is assigned a sixth ability score, which is lower than the fifth ability score in step 122. In some embodiments, the sixth ability score is 2. In some embodiments, the third threshold percentage is about 25%. In some embodiments, the third threshold percentage is in the range of about 15% to about 40%, including any percentage comprised therein.

If the ability analysis device 18 determines that the subject did not complete more than the third threshold percentage of the physical task in step 118, the ability analysis device 18 is configured to classify the subject ability as ‘complete assistance’ in step 124, and the subject is assigned a seventh, which is lower than the sixth ability score in step 126. In some embodiments, the seventh ability score is 1.

In some embodiments, the ability analysis device 18 can assign different ability scores for different threshold percentages of task completed by the subject, with or without the aid of a helper 26′ or assistive device 28. In some embodiments, the ability analysis device 18 can assign more or fewer ability scores than those described earlier. In some embodiments, the ability monitoring system 10 can detect more than one helper 26′ and/or assistive device 28.

Based on the subject ability score that is transmitted to the user device, the care giver may recommend physical therapy, identify problem areas for therapy, recommend subject re-positioning to prevent pressure ulcers, identify potential problem areas for pressure ulcers to engage proactive care, or initiate closer tracking of subject improvement that enables an accurate understanding of the impact of treatment on subject ability.

Although this disclosure refers to specific embodiments, it will be understood by those skilled in the art that various changes in form and detail may be made without departing from the subject matter set forth in the accompanying claims.

1. A method for monitoring subject ability comprising the steps of:
transmitting, using a transmitting device, a signal from a subject performing a task, the signal indicating subject ability to be independent,
acquiring, using a receiving device, data transmitted from the signal,
processing the data by an ability analysis device, the ability analysis device configured to calculate a subject ability score,
identifying a treatment protocol based on the subject ability score, and
outputting the subject ability score and the treatment protocol to a user device.

2. The method of claim 1, wherein the method further comprises:
identifying an assistive device in a vicinity of the subject, and
detecting use of the assistive device by the subject when the subject is performing the task.

3. The method of claim 2, wherein the method further comprises:
identifying a helper in a vicinity of the subject, and
detecting assistance provided by the helper when the subject is performing the task.

4. The method of claim 3, wherein if the subject does not use the assistive device and does not use the assistance provided by the helper to perform the task, the subject is assigned a first ability score.

5. The method of claim 4, wherein if the subject uses the assistive device and does not use the assistance provided by the helper to perform the task, the subject is assigned a second ability score lower than the first ability score.

6. The method of claim 5, wherein if the assistance provided by the helper is for safety purposes and the subject independently performs about 100% of the task, the subject is assigned a third ability score lower than the second ability score.

7. The method of claim 6, wherein if the assistance provided by the helper is for light touching and the subject independently performs about 100% of the task, the subject is assigned a fourth ability score lower than the third ability score.

8. The method of claim 7, wherein if the subject independently performs more than a first threshold percentage of the task and less than about 100% of the task, the subject is assigned the fourth ability score lower than the third ability score.

9. The method of claim 8, wherein if the subject independently performs more than a second threshold percentage of the task and less than the first threshold percentage of the task, the subject is assigned a fifth ability score lower than the fourth ability score.

10. The method of claim 9, wherein if the subject independently performs more than a third threshold percentage of the task and less than the second threshold percentage of the task, the subject is assigned a sixth ability score lower than the fifth ability score.

11. The method of claim 10, wherein if the subject independently performs less than the third threshold percentage of the task, the subject is assigned a seventh ability score lower than the sixth ability score.

12. A system for monitoring subject ability comprising:
a transmitting device for transmitting a signal from a subject performing a task, the signal indicating subject ability to be independent,
a receiving device for acquiring data from the signal,
an ability analysis device for processing the data, the ability analysis device configured to calculate a subject ability score and to identify a treatment protocol based on the subject ability score, and
a user device for outputting the subject ability score and the treatment protocol to a user.

13. The system of claim 12, further comprising an assistive device in a vicinity of the subject, the assistive device configured to transmit information to the ability analysis device.

14. The system of claim 12, wherein the ability analysis device is configured to identify a helper in a vicinity of the subject, and to detect assistance provided by the helper when the subject is performing the task.

15. The system of claim 14, wherein the ability analysis device is configured to determine if the helper is a caregiver or a member of the subject family.

16. The system of claim 14, wherein the assistive device is configured to track contact points to predict pressure ulcers on the subject.

17. The system of claim 14, wherein the ability analysis device is configured to assign an ability score to the subject based on the use of the assistive device and the helper by the subject.

18. An ability analysis device configured to process ability data of a subject obtained from a receiving device, calculate a subject ability score of the subject based on the processed data, and output the subject ability score to a user device.

19. The device of claim 18, wherein the receiving device is configured to receive the data from a transmitting device.

20. The device of claim 19, wherein the device is further configured to communicate with an assistive device in a vicinity of the subject, the assistive device configured to transmit information to the ability analysis device.
